(19) United States
(12) Patent Application Publication
(10) Pub. No.: US 2003/0232097 A1
(43) Pub. Date: Dec. 18, 2003

(54) Oily wax matrix suspension formulation comprising ibuprofen free acid and potassium salt of ibuprofen

(75) Inventors: Ramachandran Radhakrishnan, Bangalore (IN); Nehru Babu Gaddipati, Somerset, NJ (US)

Correspondence Address: VOLPE AND KOENIG, P.C., UNITED PLAZA, SUITE 1600, 30 SOUTH 17TH STREET, PHILADELPHIA, PA, 19103, US

(73) Assignee: Strides Inc., Somerset, NJ

(21) Appl. No.: 10/288,110

(22) Filed: Nov. 5, 2002

(30) Foreign Application Priority Data
Jun. 17, 2002 (IN) 649/DEL/2002
Jun. 17, 2002 (IN) 650/DEL/2002

(51) Int. Cl. A61K 31/192; A61K 9/14; A61K 35/78

(52) U.S. Cl. 424/731; 514/570; 424/727; 424/750; 424/756; 424/757; 424/765; 424/769; 424/776; 424/735

(57) ABSTRACT

An oily wax matrix suspension pharmaceutical formulation is prepared for oral administration of an active ingredient, such as ibuprofen, through a soft gelatin capsule drug delivery device. The ibuprofen may be in free acid or a mixture of free acid and ibuprofen alkali salt form. The active pharmaceutical ingredient is embedded in an oily wax matrix which is preferably blended with a surfactant. A preferred surfactant is lecithin, a preferred suspending agent is yellow beeswax, and a preferred suspension medium is soybean oil which are all natural ingredients.

### FIELD OF THE INVENTION

This invention in general relates to suspension formulations of hydrophobic non-steroidal drugs and in particular to an orally administrable oily wax matrix suspension formulation of ibuprofen, encapsulated into soft gelatin capsules.

### BACKGROUND OF THE INVENTION

Ibuprofen, namely 2-(4-isobutylphenyl) propionic acid is a wellknown medicament with analgesic, anti-inflammatory and anti-pyretic properties. Ibuprofen is available under prescription and OTC (e.g. Motrin, Brufen), primarily for the treatment of painful and anti-inflammatory disorders including rheumatoid arthritis, ankylosing spondylitis, osteoarthritis, postoperative pain, post-partum pain and soft tissue injuries, generally at doses of up to 3200 mg per day. Ibuprofen is a white powder or crystal, which is sparingly soluble in water.

U.S. Pat. No. 6,231,890 to Natio et al., describes a suspension of sparingly water-soluble acidic drug ibuprofen. Suspension comprising xanthan gum and crystalline cellulose and other are polyglycerol fatty acid ester, a water-soluble polyhydric alcohol, some inorganic compound from the group consisting of magnesium compound, aluminum compound, titanium compound and silicic acid compound.

U.S. Pat. No. 5,712,310 to Koch describes a substantially water insoluble drug ibuprofen being suspended in a formulation including a first suspending agent 0.5% and about 0.7% w/v, second suspending agent 0.1% and about 1.0% w/v, wetting agent 0.05% and about 0.2% w/v, sweetener 20% and about 40% w/v, humectant 1% and about 40% w/v, and antimicrobial agent 0.05% and about 0.5% w/v.

U.S. Pat. No. 5,621,005 to Gowan, Jr., describes an aqueous ibuprofen suspension composition comprising xanthan gum, pre-gelatinized starch, sucrose, glycerin sorbitol, polyoxyethylene sorbitan monooleate, citric acid and water.

U.S. Pat. No. 6,245,355 to Baichwal describes a sustained release ibuprofen dosage form comprising xanthan gum and a cross linking agent, locust bean gum.

European Patent Application No. EP0439344 describes an ibuprofen-containing, topical, hydro-alcoholic gel for treating inflammation or pain. Corresponding U.S. Pat. No. 5,093,133 describes an ibuprofen-containing, topical, hydro-alcoholic gel for treating inflammation or pain, methods for delivering ibuprofen through the skin to treat inflammation or pain using this gel; and using (S)-ibuprofen topically to treat inflammation or pain.

Ibuprofen is also available in salt form. Pharmaceutically acceptable salts of ibuprofen useful in practicing the present invention include alkali metal salts such as potassium and sodium salts, alkaline earth salts such as calcium or magnesium, amine salts such as tert-butyl amine, ethyl amine, triethanolamine, etc., group 13 salts such as aluminum, and amino acid salts such as lysine, argeinine, and glycine.

U.S. Pat. No. 4,361,580 to Peck et al., describes a pharmaceutical liquid composition of the aluminum salt of ibuprofen comprising of microcrystalline cellulose, sodium carboxy methyl cellulose, magnesium aluminum silicate powder and sucrose, fructose, glucose or mixture thereof.

U.S. Pat. No. 5,262,179 to Gregory et al., describes effervescent water soluble composition of ibuprofen salt in which unpleasant taste of salt is masked by the taste masking agent, comprising an alkali metal carbonate, alkali metal monohydrogen phosphate or alkali metal tribasic citrate.

U.S. Pat. No. 4,859,704 to Haas describes a pharmaceutically and commercially acceptable dosage form of alkali salt of ibuprofen comprising potassium salt of ibuprofen and the alkali metal bicarbonate dissolved in the aqueous medium.

U.S. Pat. No. 4,859,704 to Haas describes a novel water soluble alkali metal salt of ibuprofen is prepared by reacting ibuprofen and alkali metal bicarbonate in aqueous medium. It permits the preparation of a number of novel formulations, which result in pharmaceutically acceptable dosages forms, such as tablets, capsules, liquids or parenterals.

U.S. Pat.. No. 6,210,710 to Skinner describes a pharmaceutical composition comprising ibuprofen, a blend and the lubricating agents like stearic acid, colloidal silicon dioxide, magnesium stearate, calcium stearate, waxes, polyethylene glycol, and magnesium lauryl sulfate.

The patient compliance is improved if soft gelatin capsule is used for drug administration, because of its soft, elastic character making it easier to swallow compared to conventional tablets or hard gelatin capsules.

Furthermore, since the dosage form is generally swallowed, it is unnecessary to flavor or otherwise mask any unpleasant taste of the active pharmaceutical ingredients. Finally, unlike tablets, soft gelatin capsules do not chip or powder.

Filled one-piece softgels have been widely known and used for many years and for a variety of purposes. Because softgels have properties, which are different from conventional two-piece hard gelatin capsules, the softgels are capable of retaining liquid fill material. Typically, softgels are used to contain orally consumable materials such as vitamins and pharmaceutical compositions in a liquid vehicle or carrier.

In general, not all liquids are suitable as vehicles or carriers for inclusion in softgels. For example, water, propylene glycol, glycerin, low molecular weight alcohols, ketones, acids, amines and esters cannot be used as a carrier in softgels by themselves since they interact with the gel and, if present, they can only be present in relatively small amounts.

Another limitation associated with softgels is the ability to incorporate a single dose of the pharmaceutically active ingredient in solution in an acceptable fill volume. Often, it is difficult to dissolve the pharmaceutically active ingredient in a volume of solvent small enough to produce a softgel, which delivers the desired dosage amount, is economically appropriate and comfortable to ingest by the patient. Developing solvent systems for pharmaceutically active ingredients that neither significantly interacts with the active ingredient nor the softgel casing it self, has proven a difficult art.

The chemical properties of certain types of drugs have necessitated the development of special solvent systems for softgel dosage forms. Yu et al., Australian Patent Application No. 81573/87 discloses pharmaceutical formulations suitable for filling softgels comprising acidic pharmaceutical agents and solvent systems, the solvent systems comprising 10% to 80% by weight polyethylene glycol, 1% to 20% by weight water and hydroxide ion species. The solvent systems dissolve the pharmaceutical agent, e.g., ibuprofen, in concentrations sufficient for use in soft gelatin capsules.

U.S. Pat. No. 6,251,426 to Gullapalli describes a liquid softgel fill formulation consisting ibuprofen in free acid with polyethylene glycol and polyvinylpyrrolidone.

U.S. Pat. 6,027,746 to Lech describes a liquid oral suspension incorporated into a softgel capsule wherein the decongestant is selected from a group comprising of certain pharmaceutical actives. This disclosure is also addressed at an active agent consisting of a particulate adsorbate. The drug delivery device is typically a chewable gelatin capsule.

U.S. Pat. No. 4,690,823 to Lohner et al., describes a process for manufacturing ibuprofen-containing soft gelatin capsules in which up to 30 parts by weight of ibuprofen in free form is dissolved in from 70 to 85 parts by weight of polyoxyethylene-polyoxypropylene polymer or in a mixture of from 30 to 76 parts by weight of a polyalkylene glycol and from 7 to 40 parts by weight of a surfactant having a very rapid and high bio-availability of the active ingredient. Preferred suitable surfactants include, for example, polyoxyethyleneglycerol trihydroxystearate, polyoxyethylene (C₁₂-C₁₈)-fatty alcohol ethers, polyoxyethylene stearate, polyoxyethylene sorbitan mono (C₁₂-C₁₈)-fatty acid esters, and polyoxyethylene-polyoxypropylene polymer.

In the Lohner et al. process, up to 30 parts by weight of free form ibuprofen is dissolved by heating the free form ibuprofen with the selected solvent at a temperature of 45° to 60° C. If a concentration of ibuprofen greater than the maximum 30 parts by weight possible in solution is desired, up to 40 parts additional parts by weight of ibuprofen may also be suspended in the fill.

Softgel fills containing high concentrations of ibuprofen as a mixture of ibuprofen in free form and its salts are described in U.S. Pat. Nos. 5,071,643 and 5,360,615 to Yu et al. These fill formulations may be liquid, semi-solid or solid, and are formed by mixing 40-80% by weight ibuprofen, 0.1-1.5 moles of hydroxide ion per mole of ibuprofen, 1-20% by weight water, and 4-12% by weight glycerin or propylene glycol in polyethylene glycol. Solubility of the ibuprofen salts is further enhanced 2-10% by the further addition of 3-10% by weight of glycerin, or propylene glycol or 1-20% by weight of polyvinylpyrrolidone.

The preferred average molecular weight of the polyvinylpyrrolidone is 10,000-100,000. Higher percentages of above 5% polyvinylpyrrolidone, and higher molecular weight polyvinylpyrrolidone are used to prepare semi-solid and solid formulations for suppositories, two-piece capsules, and tablets.

Upon mixing under the conditions described by Yu, et al., the polyethylene glycol acts to dissolve the free form of the ibuprofen; the hydroxyl ions source, e.g., sodium hydroxide or potassium hydroxide, partially forms an ibuprofen salt, and the water forms a salvation sphere around the acid salt permitting it to go into solution in the polyethylene glycol.

U.S Pat. No. 5,376,688 to Morton et al, describes a pharmaceutically acceptable solution of acidic, basic or amphoteric pharmaceutical agent like ibuprofen, ketoprofen, naproxen, ranitidine Pseudoephedrine suitable for encapsulation in gelatin capsule for subsequent oral administration a comprising a pharmaceutical agent and a solvent system consisting essentially of a solvent selected from the group consisting of diethylene glycol monoethyl ether, polyglycerol oleate and mixture thereof.

A composition including soybean oil, yellow beeswax and lecithin is disclosed in U.S. Pat. No. 6,309,677 to Gorenbein et al. Cartenoids are mixed with these excipients to form a dietary supplement.

U.S. Pat. No. 5,175,002 to Torosian provides for a suspension formulation comprising amantadine hydrochloride in soybean oil, lecithin and wax.

U.S. Pat. No. 6,096,338 to Lacy et al. discloses a carrier system for a hydrophobic drug. The carrier system described comprises a digestible oil and a pharmaceutically acceptable surfactant having hydrophilic and lipophilic components.

U.S. Pat. No. 5,672,358 to Tabibi et al. discloses a controlled release aqueous emulsion, a method of manufacture thereof and use as a vehicle for delivering medicaments in liquid form of Ibuprofen active. Emulsion is oil-in-water emulsion, oil and water phases being emulsified by a phospholipid emulsifier that is lecithin admixed with ethoxylated mono-diglycerides and propylene glycol. The said oil phase comprises a wax matrix (beeswax) that is a wax having a melting point of about 40° to about 80°.

### SUMMARY OF THE INVENTION

A soft gelatin capsule formulation containing ibuprofen free acid or a combination of free acid and ibuprofen alkali metal salt is provided, particularly a suspension formulation of natural ingredients, preferably without any solubilizer or synthetic polymers. A soft gelatin capsulated formulation provides better patient compliance. A suspension formulation provides stability of the drugs over prolonged period of time, and also offers uniformity to the active drug. Further increase in the viscosity of the solid drug is achieved by using a suspending agent. Suspension formulations often use a suspending agent to minimize particle sedimentation during storage. Preferably, the alkali metal salt of ibuprofen is formed by the reaction of ibuprofen and potassium carbonate in aqueous solution.

In accordance with one preferred embodiment, soft gelatin capsules of a pharmaceutical formulation comprising 200 mg by weight of ibuprofen containing a mixture of both free acid and alkali metal salt, 1-15 mg by weight of yellow beeswax, 5-30 mg by weight of lecithin 0.5 to 15 mg of potassium carbonate, 100-300 mg by weight of soybean oil and 1 to 200 μL of purified water are provided.

In accordance with another preferred embodiment, there are provided soft gelatin capsules of a pharmaceutical formulation comprising 200 mg by weight of ibuprofen in free acid form, 1-15 mg by weight of yellow beeswax, 5-35 mg by weight of lecithin and 100-300 mg by weight of soybean oil.

In accordance with another preferred embodiment, a method of making a pharmaceutical formulation is provided which entails preparing an oily matrix of soybean oil and beeswax, blending lecithin to the oily matrix, blending potassium carbonate solution to the oily matrix containing soybean oil, lecithin and beeswax, mixing an active pharmaceutical ingredient into the matrix, and enclosing the oily matrix embedded pharmaceutical complex into a capsule, wherein preferably 200 mg of ibuprofen having a mixture of free acid and potassium salt is used as the active pharmaceutical ingredient for each capsule.

In accordance with another preferred embodiment, a methods of making a pharmaceutical formulation is provided which entails preparing an oily matrix of soybean oil and beeswax, blending lecithin to said oily matrix, lecithin and beeswax, mixing an active pharmaceutical ingredient into the matrix, and enclosing the oily matrix embedded pharmaceutical complex into a capsule, wherein preferably 200 mg of ibuprofen free acid is used as the active pharmaceutical ingredient for each capsule.

### DETAILED DESCRIPTION OF THE PREFERRED EMBODIMENT

The present invention relates to pharmaceutical formulations comprising i)ibuprofen free acid, and ii)a mixture of ibuprofen salt and free acid for oral administration in the form of soft gelatin capsules. The formulations also preferably comprise of yellow beeswax, soybean oil, lecithin, potassium carbonate and purified water. Natural ingredients are used in the preferred embodiment such as soybean oil as a suspension medium, lecithin as a surfactant and yellow beeswax as a suspension agent. Preferably, potassium carbonate and purified water are used to form the potassium salt of ibuprofen.

Soybean oil is a pale yellow to brownish yellow liquid derived from soybeans either by solvent extraction or by expression, containing triglycerides of oleic acid, linoleic acid and linollenic acid. It is used in the formulation as a diluent and suspension medium. Other examples of suspension medicine that may. be used include almond oil, babassu oil, borage oil, blackcurrant seed oil, canola oil, castor oil, coconut oil, corn oil, cottonseed oil, evening primrose oil, grape seed oil, groundnut oil, mustard seed oil, olive oil, palm oil, palm kernel oil, peanut oil, rapeseed oil, safflower oil, sesame oil, shark liver oil, sunflower oil, hydrogenated castor oil, hydrogenated coconut oil, hydrogenated palm oil, hydrogenated soybean oil, hydrogenated vegetable oil, hydrogenated cottonseed and castor oil, partially hydrogenated soybean oil, soy oil, glyceryl tricaproate, glyceryl tricaprylate, glyceryl tricaprate, glyceryl triundecanoate, glyceryl trilaurate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, saturated polyglycolized glycerides, linoleic glycerides, caprylic/capric glycerides, modified triglycerides, fractionated triglycerides, and mixtures thereof.

Beeswax is obtained from honeycombs and is made of esters of straight-chain monohydric alcohols with even numbered carbon chains from C₂₄ to C₃₆ esterified with straight-chain acids also having even numbers of C atoms up to C₃₆. Beeswax is yellow to brownish-yellow, practically insoluble in water, but soluble in organic solvents such as chloroform. Beeswax is used as a suspending agent in the formulation. Other natural and synthetic waxes, paraffin, carnauba wax, petroleum wax, white or yellow beeswax, castor wax, candelilla wax, rice bran wax, microcrystalline wax, may be used.

Lecithin is phosphatidylcholine containing a mixture of diglycerides of stearic, palmitic, and oleic acids, linked to the choline ester phosphoric acid. Lecithin is used in the formulation as a surfactant, and suspension stabilizer. Lecithin also provides lubricity to the product and facilitates the flow of the suspension from the hopper. Lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, chenodeoxycholate, lithocholate, ursodeoxycholate, taurocholate, glycocholate, deoxycholate, taurodeoxycholate, chenodeoxycholate, glycodeoxycholate, glycochenodeoxycholate, taurochenodeoxycholate, ursodeoxycholate, lithocholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, tetra acetyl sulfate, docusate, lauroyl carnitine, palmitoyl carnitine, myristoyl carnitine, and salts and mixtures thereof.

Potassium Carbonate: K₂CO₃, is used to form the potassium salt of ibuprofen. Other carbonates may be used in place of potassium carbonate. These include alkaline metal carbonates (such as sodium), alkaline earth carbonates (such as calcium, magnesium or barium), amine carbonates, Group 13 carbonates (such as aluminum carbonates), and amino acid carbonates.

The following examples illustrate the preferred embodiments of pharmaceutical compositions comprising ibuprofen free acid and/or ibuprofen salt as principal active ingredient.

### EXAMPLES

### Example 1

| Fill Ingredients | Composition by weight (mg) |
|---|---|
| Ibuprofen | 200 |
| Yellow Beeswax | 1-15 |
| Lecithin, NF | 5-30 |
| Potassium Carbonate | 0.5-15 |
| Soybean Oil, USP | 100-300 |
| Purified Water | 1-200 |

The fill was prepared by heating the soybean oil to 60°-65° C. The yellow beeswax was added and mixed until the wax was melted and the dispersion was homogenous. This was followed by lecithin addition. Potassium carbonate was dissolved in purified water and the solution was slowly added to the soybean oil—beeswax—lecithin mixture until homogenous. This mixture was continued to be stirred, and ibuprofen was mixed in to form a homogenous dispersion. Finally, the blend was deaerated to remove any trapped gases

### Example 2

| Ingredients | Composition by weight | |
|---|---|---|
| Ibuprofen | 200 | mg |
| Yellow Beeswax | 1-15 | mg |
| Lecithin, NF | 5-35 | mg |
| Soybean Oil, USP | 100-300 | mg |

The fill was prepared by heating the soybean oil to 60°-65° C. The yellow beeswax was added and mixed until the wax was melted and the dispersion was homogenous. This was followed by lecithin addition. This mixture was continuously stirred, and ibuprofen was mixed in to form a homogenous dispersion. Finally, the blend was deaerated to remove any trapped gases.

In general, gelatin capsule sheath formulations for soft gelatin capsules comprise raw gelatin and one or more plasticizers added to adjust the hardness of the capsule. Typical plasticizers include glycerin, sorbitol and Anidrisorb 85/70. A preferred plasticizer is Anidrisorb 85/70, an aqueous solution of D-sorbitol and sorbitans. One preferred gelatin formulation for the soft gelatin capsules used in accordance with preferred embodiments includes gelatin in the range of about 40% to 48% and a plasticizer ranging in amount from about 16% to 35%. Another preferred plasticizer is sorbitol, a non-crystallizing sorbitol solution. When either a 70% non-crystallizing sorbitol solution or Anidrisorb™ 85/70 is used alone, the amount of plasticizer used preferably ranges from about 16% to 35%. Capsule formulations can also include other suitable additives such as anti-oxidants, amino acids and coloring agents, which impart specific characteristics including capsule aesthetics. Anti-oxidants include Butylated Hydroxy Anisole (BHA), Butylated Hydroxy Toluene (BHT), and citric acid, though other antioxidants such as tocopherol, tocopherylacetate, d-α-tocopheryl polyethylene glycol 1000 succinate, cysteine, ascorbic acid, calcium propionate, sorbic acid, potassium sorbate, ethoxyquin, lactic acid, benzoic acid, sodium benzoate, ethyl-p-hydroxybenzoate, and propyl-p-hydroxybenzoate may be used. FD&C dyes and D & C dyes are examples of pharmaceutically acceptable coloring agents that may be used in preferred embodiments.

The formulation is a suspension containing either free acid or a mixture of free acid and potassium salt in an oily wax matrix. The solubility is not a limiting factor and the same concept can be extended to higher strengths. In the first example, each soft gel gap seal contains 200 mg of ibuprofen. However, other strengths including, but not limited to, 400, 600 and 800 mg products can be produced.

Gelatin paste preparation is preferably carried out in a melter. The gelatin paste preparation is done by heating the gelatin with plasticizer and purified water with continuous stirring. During gelatin paste preparation, vacuum is applied to remove extra amounts of water added and to get a gelatin ribbon free from air bubbles. Colorants may be optionally added and mixed further in a stainless steel tank at 60±5° C. for 1 to 2 hours to get a uniform color distribution. The blend of the fill and gelatin paste as obtained above is further taken for encapsulation. Manufacturing of soft gelatin capsules is carried out preferably using rotary die process. However other processes like plate process, Accogel™ process and bubble method may be used. The shape of capsule may be oval, round or oblong, most preferably oval shaped with a 16 mm length. Encapsulation process is preferably carried out at temperature below 30° C. and relative humidity below 25%.

The following table of Examples 3-6 illustrate preferred embodiments of several soft-gelatin-shell ibuprofen formulations. These examples illustrate particular embodiments of the invention and are not intended to limit the scope of the invention in any way.

Examples

| | 3 | 4 | 5 | 6 |
|---|---|---|---|---|
| Ingredients | Weight percent range (min-max) | | | |
| Gelatin | 38.0-46.0 | 38.0-46.0 | 38.0-46.0 | 38.0-46.0 |
| Sorbitol Solution | 14.0-25.0 | 14.0-25.0 | 14.0-25.0 | 14.0-25.0 |
| Glycine | 0.2-0.6 | 0.2-0.6 | 0.2-0.6 | 0.2-0.6 |
| BHA | 0.02-0.03 | 0.02-0.03 | 0.02-0.03 | 0.02-0.03 |
| BHT | | 0.02-0.03 | | 0.02-0.03 |
| Citric Acid | | | 0.42-0.46 | 0.42-0.46 |
| Purified water | 40.5-45.5 | 40.5-45.5 | 40.5-45.5 | 40.5-45.5 |

Certain modifications and improvements of the disclosed invention will occur to those skilled in the art without departing from the scope of invention, which is limited only by the appended claims.

What is claimed is:

1. An orally administrable pharmaceutical formulation comprising:an active pharmaceutical ingredient of ibuprofen embedded into an oily wax matrix; the oily wax matrix comprising:
a surfactant;
a wax suspending agent; and
an oil suspension medium.

2. The orally administrable pharmaceutical formulation according to claim 1 wherein the formulation is a suspension formulation and the surfactant, wax suspending agent and oil suspension medium are all natural ingredients.

3. The orally administrable pharmaceutical formulation according to claim 1 wherein the surfactant is lecithin.

4. The orally administrable pharmaceutical formulation according to claim 1 wherein the suspending agent is yellow beeswax.

5. The orally administrable pharmaceutical formulation according to claim 1 wherein the said suspension medium is selected from the group comprising:
almond oil, babassu oil, borage oil, blackcurrant seed oil, canola oil, castor oil, coconut oil, corn oil, cottonseed oil, evening primrose oil, grape seed oil, groundnut oil, mustard seed oil, olive oil, palm oil, palm kernel oil, peanut oil, rapeseed oil, safflower oil, sesame oil, shark liver oil, sunflower oil, hydrogenated castor oil, hydrogenated coconut oil, hydrogenated palm oil, hydrogenated soybean oil, hydrogenated vegetable oil, hydrogenated cottonseed and castor oil, partially hydrogenated soybean oil, soy oil, glyceryl tricaproate, glyceryl tricaprylate, glyceryl tricaprate, glyceryl triundecanoate, glyceryl trilaurate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, saturated polyglycolized glycerides, linoleic glycerides, caprylic/capric glycerides, modified triglycerides, fractionated triglycerides, and mixtures thereof.

6. The orally administrable pharmaceutical formulation according to claim 1 wherein the suspension medium is soybean oil.

7. The orally administrable pharmaceutical formulation according to claim 1 wherein the active pharmaceutical ingredient is ibuprofen in free acid form, the surfactant is lecithin, the suspending agent is beeswax and the suspension medium is soybean oil.

8. The orally administrable pharmaceutical formulation according to claim 7 wherein the formulation is disposed in a soft gelatin capsule.

9. The orally administrable pharmaceutical formulation according to claim 7, the formulation comprising:
about 200 mg by weight of ibuprofen;
about 1-15 mg by weight of yellow beeswax;
about 5-35 mg by weight of lecithin; and
about 100-300 mg by weight of soybean oil.

10. The orally administrable pharmaceutical formulation according to claim 9 wherein the formulation is disposed in a soft gelatin capsule.

11. The orally administrable pharmaceutical formulation according to claim 1 wherein the active pharmaceutical ingredient is mixture of ibuprofen free acid and in an alkali metal salt form, the surfactant is lecithin, the suspending agent is beeswax, and the suspension medium is soybean oil.

12. The orally administrable pharmaceutical formulation according to claim 11 wherein the salt forming agent is Potassium Carbonate.

13. The orally administrable pharmaceutical formulation according to claim 11 wherein the formulation is disposed in a soft gelatin capsule.

14. The orally administrable pharmaceutical formulation according to claim 11 comprising:
about 200 mg by weight of Ibuprofen salt;
about 1-15 mg by weight of yellow beeswax;
about 5-30 mg by weight of lecithin;
about 100-300 mg by weight of soybean oil;
about 0.5-15 mg by weight of Potassium Carbonate; and
about 1-200 μl Purified Water.

15. The orally administrable pharmaceutical formulation according to claim 14 wherein the formulation is disposed in a soft gelatin capsule.

16. A process for producing an orally administrable pharmaceutical formulation, the process comprising:
preparing an oily wax matrix of soybean oil and beeswax;
blending lecithin into the oily wax matrix;
mixing an active pharmaceutical ingredient of ibuprofen into the matrix; and
encapsulating the oily matrix-embedded pharmaceutical blend into a capsule.

17. The process for producing an orally administrable pharmaceutical formulation according to claim 16 wherein the formulation is a suspension formulation.

18. The process for producing an orally administrable pharmaceutical formulation according to claim 16 wherein the capsule is a soft gelatin capsule.

19. The process for producing an orally administrable pharmaceutical formulation according to claim 16 further comprising:
heat treating soybean oil at 60°-65° C.;
adding beeswax to the soybean oil;
stirring the resultant mixture until the wax is melted, and dispersed homogenously;
adding lecithin into the mixture;
adding ibuprofen in free acid form into the mixture, while stirring to form a homogenous blend thereof;
deareating the blend to remove any trapped gases; and
then encapsulating the blend into a capsule.

20. The process for producing an orally administrable pharmaceutical formulation according to claim 19 wherein the formulation is a suspension formulation.

21. The process for preparing an orally administrable pharmaceutical formulation according to claim 19 wherein the capsule is a soft gelatin capsule.

22. The process for producing an orally administrable pharmaceutical formulation according to claim 16 further comprising:
heat treating soybean oil at 60°-65° C.;
adding beeswax to the soybean oil;
stirring the resultant mixture until the wax is melted and dispersed homogenously;
adding lecithin into the mixture;
adding potassium carbonate which has been dissolved in purified water to the mixture;
adding ibuprofen into the mixture, while stirring to form a homogenous blend thereof;
deareating the blend to remove any trapped gases; and
then encapsulating the blend into a capsule.

23. The process for preparing an orally administrable pharmaceutical formulation according to claim 22 wherein the capsule is a soft gelatin capsule.

24. A process for producing an orally administrable pharmaceutical formulation, the process comprising:
preparing an oily wax matrix of a wax suspending agent and an oil suspension medium;
blending a surfactant into the oily wax matrix;
mixing an active pharmaceutical ingredient of ibuprofen into the matrix; and
encapsulating the oily matrix-embedded pharmaceutical blend into a capsule.

25. The process for producing an orally administrable pharmaceutical formulation according to claim 24 wherein the surfactant, wax suspending agent and oil suspension medium are all natural ingredients.
